(19) United States
(12) Patent Application Publication
(10) Pub. No.: US 2022/0031660 A1
(43) Pub. Date: Feb. 3, 2022

(54) CANCER TREATMENT COMPOSITION

(71) Applicant: BOTANICAL RESOURCE HOLDINGS (PROPRIETARY) LIMITED, Somerset West (ZA)

(72) Inventor: Richard Paul DAVIES, Somerset West (ZA)

(21) Appl. No.: 17/504,585

(22) Filed: Oct. 19, 2021

Related U.S. Application Data
(63) Continuation-in-part of application No. 15/562,069, filed on Sep. 27, 2017, now Pat. No. 11,173,142.

(30) Foreign Application Priority Data
Mar. 27, 2015 (ZA) 2015/02137

(51) Int. Cl. A61K 31/404

(52) U.S. Cl. CPC A61K 31/404

(57) ABSTRACT

The present invention relates to anti-cancer compositions based on extracts of a plant from the Mesembryanthemaceae family, in particular Mesembryanthemum tortuosum (Sceletium tortuosum), their use in the treatment of cancer, and methods of manufacturing the compositions. The anti-cancer compositions of the present invention include Δ7 mesembrenone.

### BACKGROUND OF THE INVENTION

THIS invention relates to anti-cancer compositions based on extracts of a plant from the Mesembryanthemaceae family, in particular Mesembryanthemum tortuosum (Sceletium tortuosum), their use in the treatment of cancer, and methods of manufacturing the compositions.

Mesembryanthemum tortuosum, or Sceletium tortuosum as it is more commonly referred to in modern times, has been used for many centuries by indigenous peoples of Southern Africa, most notably the southern parts of the Western Cape and Namaqualand, and its use has been recorded in the literature for over 300 years. When prepared for chewing, typically by crushing of selected parts of the plant material, fermentation and drying, the resultant product, known locally as “kougoed” (stuff to chew), “Channa” or “Karma”, is said to have mood enhancing and stimulant properties, and even pain and hunger relieving properties.

US patent publication 2012/0004275 discloses compositions including as active ingredient an extract of a plant of the family Mesembryanthemaceae with mesembrenol and mesembrenone as the two major alkaloids present and to their use as PDE4 inhibitors.

### SUMMARY OF THE INVENTION

According to a first aspect of the invention, there is provided an extract of a plant or plants from the Mesembryanthemaceae family, such as Mesembryanthemum tortuosum, having an alkaloid profile comprising at least 20%, or at least 50%, or at least 70% (w/w) Δ7 mesembrenone.

According to a preferred embodiment of this aspect of the invention, the alkaloid profile comprises at least 70% (w/w) Δ7 mesembrenone. Preferably the majority of the balance of the alkaloids is mesembrine and mesembrenone.

In one form of this aspect of the invention the extract has an alkaloid profile comprising at least about 70% (w/w) Δ7 mesembrenone, about 20% (w/w) mesembrine and about 10% (w/w) mesembrenone.

The total alkaloid content of the extract is typically at least 10%, or at least 15%, or at least 20% thereof.

According to a second aspect of the invention, there is provided a method of treating cancer comprising administering to a patient in need of such treatment a therapeutically effective amount of an extract of a plant or plants from the Mesembryanthemaceae family, such as Mesembryanthemum tortuosum, having an alkaloid profile comprising at least 20%, or at least 50%, or at least 70% (w/w) Δ7 mesembrenone.

According to a third aspect of the invention, there is provided an extract of a plant or plants from the Mesembryanthemaceae family, such as Mesembryanthemum tortuosum, having an alkaloid profile comprising at least 20%, or at least 50%, or at least 70% (w/w) Δ7 mesembrenone for use in a method of treating cancer in a patient in need of such treatment.

According a fourth aspect of the invention, there is provided a composition comprising an alkaloid content/profile as hereinbefore defined.

According to a fifth aspect of the invention, there is provided an anti-cancer composition comprising Δ7 mesembrenone, mesembrenone, or a combination of Δ7 mesembrenone and mesembrenone.

In a preferred embodiment of this aspect of the invention, the anti-cancer composition is a pharmaceutical composition comprising Δ7 mesembrenone and a pharmaceutically acceptable carrier, together with other optional pharmaceutically acceptable excipients.

In a particularly preferred embodiment of this aspect of the invention, the pharmaceutical composition comprises at least 80% pure, more preferably at least 90% pure, isolated Δ7 mesembrenone.

According to a fifth aspect of the invention, there is provided a method of treating cancer comprising administering to a patient in need of such treatment a therapeutically effective amount of Δ7 mesembrenone, mesembrenone, or a combination of Δ7 mesembrenone and mesembrenone.

In a preferred embodiment of this aspect of the invention, a therapeutically effective amount of Δ7 mesembrenone is administered to a patient in need of such treatment.

The Δ7 mesembrenone is preferably at least 80% pure, more preferably at least 90% pure, isolated Δ7 mesembrenone.

According to a sixth aspect of the invention, there is provided an anti-cancer composition comprising as active ingredient Δ7 mesembrenone, mesembrenone, or a combination of Δ7 mesembrenone and mesembrenone for use in a method of treating cancer in a patient in need of such treatment.

In a preferred embodiment of this aspect of the invention, the anti-cancer composition is a pharmaceutical composition comprising Δ7 mesembrenone and a pharmaceutically acceptable carrier, together with other optional pharmaceutically acceptable excipients.

In a particularly preferred embodiment of this aspect of the invention, the pharmaceutical composition comprises at least 80% pure, more preferably at least 90% pure, isolated Δ7 mesembrenone.

In the case of pharmaceutical compositions, in some embodiments of the invention, they are provided in unit dosage form, each unit dose comprising from about 5 mg to about 70 mg, or from about 10 mg to about 65 mg, or from about 15 mg to about 60 mg Δ7 mesembrenone.

In some embodiments of the invention, each unit dose comprises from about 1.5 mg to about 20 mg, or from about 3 mg to about 18.5 mg, or from about 4 mg to about 17.5 mg mesembrine.

In some embodiments of the invention, each unit dose comprises from about 0.75 mg to about 10 mg, or from about 1.5 mg to about 9.5 mg, or from about 2 mg to about 9 mg mesembrenone.

In some embodiments of the invention, the disease or condition responsive to treatment with an anticancer agent is with specific cytotoxicity on breast epithelial carcinoma cells (MCF-7).

In some embodiments of the invention, the cancer treated by the extracts, compositions and isolated actives (isolates) of the invention is breast cancer.

### BRIEF DESCRIPTION OF THE FIGURES

The invention will now be discussed in more detail, by way of example only, with reference to the accompanying figures in which:

FIG. 1 is an MPLC solvent gradient graph of extraction of actives from Sceletium tortuosum;

FIG. 2 is a HPLC chromatograph of an extract of the invention comprising >70% of Δ7 mesembrenone;

FIG. 3 is a bar graph depicting the results of the effect of an extract (PNP50) of Sceletium tortuosum showing the viability thereof on normal and cancerous breast epithelial cells; and

FIG. 4 is a bar graph depicting the results of the effect of isolates extracted from Sceletium tortuosum showing the viability thereof on normal and cancerous breast epithelial cells.

### DESCRIPTION OF PREFERRED EMBODIMENTS

Anti-cancer compositions of the invention comprise active ingredients derived from extracts of a plant or plants from the Mesembryanthemaceae family, such as Mesembryanthemum tortuosum, in particular of the DV-17 variety or isolated actives or combinations of isolated actives derived from the extracts.

“The DV-17 variety” is a unique variety of Mesembryanthemum (Sceletium) tortuosum selectively propagated for its high alkaloid content, recognizable profile and vigorous growth. Chemical analysis shows a distinctive fingerprint to the levels of mesembrine, mesembrenone, Δ7 mesembrenone and epimesembranol as well as other active and related compounds pre-fermentation.

Examples below are of 2 commercially available Mesembryanthemum plants showing distinct alkaloid profiles compared to the DV-17 variety.

Yield figures for mesembrine are typically between 12 mg and 15 mg per gram of dried DV-17 whole herba.

| Sample | Δ7 mesem- | Mesem- | Mesem- | Mesembrine |
|---|---|---|---|---|
| ref | brenone % | brenone % | branol % | % |
| DV-8 | | 40.2 | 3.8 | 56.0 |
| DV-12 | | 45.8 | Trace | 54.2 |
| DV-17 | <10 | ≤20 | Trace | >70 |

The composition that comprises an extract of a plant or plants from the Mesembryanthemaceae family, such as Mesembryanthemum tortuosum, as active ingredient typically has an alkaloid profile comprising at least 20% or at least 50% or at least 70% Δ7 mesembrenone. It preferably comprises at least 70% Δ7 mesembrenone, about 20% mesembrine and about 10% mesembrenone.

In preferred embodiments of the invention the anti-cancer composition comprises Δ7 mesembrenone, mesembrenone or a combination of Δ7 mesembrenone and mesembrenone. In particularly preferred embodiments of the invention, the composition comprises Δ7 mesembrenone.

The compositions of the invention may be formulated in any suitable form for pharmaceutical administration, such as for example tablets, capsules, vapes, topical balms or hydrogels, edible gummies or chewable gums, and as suppositories. The formulations may be designed for use orally, including sublingually and sub-bucally, transdermally, including via the rectum, intranasally and for delivery via the lymph system.

The pharmaceutical composition of the invention may comprise a hydro-methanolic derived extract of the plant containing desired amounts of Δ7 mesembrenone, mesembrine and mesembrenone. Accordingly, the pharmaceutical compositions, whilst derived from a natural plant material, contain a known and specified content of the active components.

### Plant Extract

Plant (Mesembryanthemum tortuosum DV-17 variety) is harvested and dried (NMT8% moisture) then milled to 250 micron. 2 kg of said plant material is added to 10 liters of methanol (82% hydro-methanolic) at 35° C. with the pH adjusted to 2.4 with hydrochloric acid then allowed to stir for 24 hrs. The crude solvent extract is removed by pressing before an additional 2 liters of methanol 88% is again added to the plant material—this time without the addition of acid—and stirred for 9 hrs before repressing.

The combined crude extract (9.8 liters) is filtered thrice through polypropylene membranes—1st 100 μm, 2nd 40 μm, and 3rd 5 μm.

7.8 liters of the main solvent (methanol) is recovered under reduced pressure—318 mb at 40° C.

The high recovery of mesembrine at this stage allows for easy oxidation to Δ7 mesembrenone; as would be evident to those skilled in the art; to increase Δ7 mesembrenone to the desired level. By way of example, the following oxidation method reported by Peter W. Jeffs (THE ALKALOIDS, Chemistry and Physiology, Volume XIX, page 55) may be used.

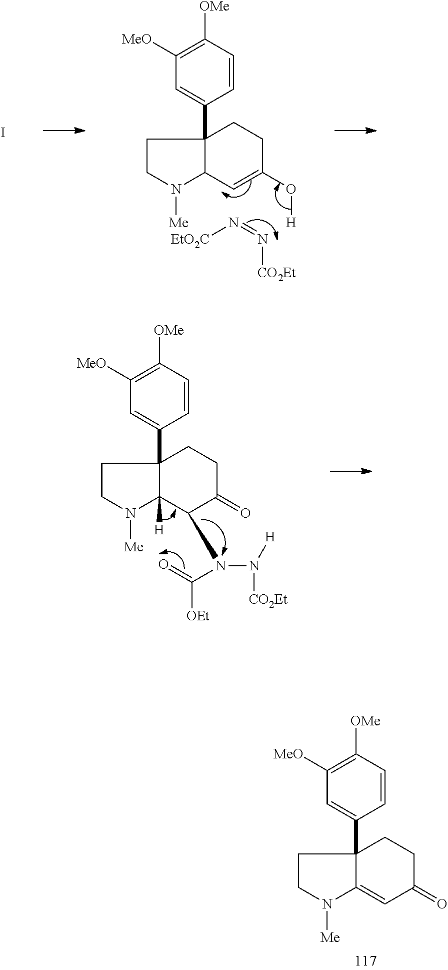

The combined aqueous phase is basified to pH 7.7 with a 25% ammonia solution and then extracted 4× with 500 mL DCM.

All organic phases are pooled and washed once with brine. After phase separation on standing the bottom organic layer is tapped off and dried with anhydrous sodium sulphate.

The solvent is removed completely under vacuum leaving a crude Sceletium extract. In order to obtain a refined extract or composition comprising at least 70% Δ7 mesembrenone, further isolation of actives was undertaken to confine the extract to the active anti-cancer agents.

### Isolation of Actives

The column is loaded onto a MPLC (Grace, Reveleris) instrument, set and run as follows:

Cartridge: Grace Reveleris 80 g Silica Cartridge

Equilibration: 3CV

Flow rate: 47 mL/min

UV threshold: 0.03 AU

UV1 wavelength: 228 nm

UV2 wavelength: 288 nm

ELSD threshold: 5 mv

ELSD carrier: Iso-proponal

Set peaks: Collect peaks

Injection type: Manual (5 g)

Solvent A: DCM

Solvent B: Methanol

Mobile phase: A:B

Run time: 16 min

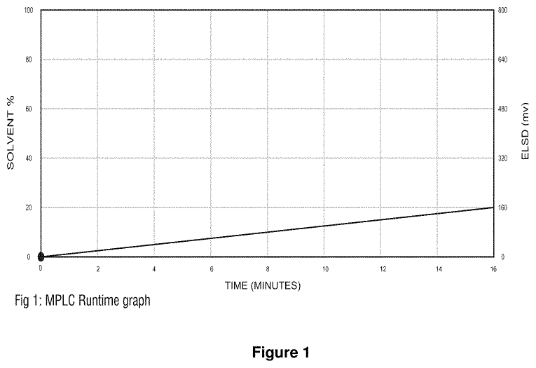

As reflected in FIG. 1 of the accompanying figures, the eluting solvent gradient is gradually changed from 0% Methanol to 20% Methanol over 16 min.

Combine fractions of interest as confirmed by TLC—Evaporate solvent under reduced pressure to obtain the purified alkaloid fractions of mesembrenone, mesembrine and Δ7 mesembrenone.

### Drying Product

Combine the following in a suitable container:

25 g alkaloid containing composition dissolved in 150 mLs of 20% Ethanol

Adjust pH to 5.1 with concentrated hydrochloric acid.

Add 75 g Dridex (DE9) to the alkaloid solution under high sheer conditions until homogenized and then place paste in a freeze dryer overnight.

Product is then reduced to a fine free flowing powder using a colloidal mill or laboratory hammer mill.

Yield: 100 g (actual 98.7 g)

Product Characteristics: Free flowing powder

Colour: Tan

Practical Size: <75 μm

Moisture Content: <3%

Total Alkaloids: NLT 21% w/w

Alkaloid Profile: 68-70% of Δ7 mesembrenone HCL

### Thin-Layer Chromatography

For qualitative screening purposes the following system is suitable: AnalTech, Inc RPS-F Silica Gel W/UV254 (250 μm layer thickness) and developed in water/methanol/ammonia solution (N7), in methanol (18:6:0.5). The plates are dried at 60° C. for 10 minutes, studied under UV254 and UV365 and then sprayed with Dragendorff's spray reagent.

(Rf of Δ7 mesembrenone=0.82)

### High Performance Liquid Chromatography (HPLC)

The alkaloids of the invention may directly be extracted in ethanol, methanol or any other suitable solvent (see, for example, http://www.ncbi.nlm.nih.gov/pubmed/21486531). For HPLC the sample has to be filtered (e.g. 0.45 μm filter) in order to protect the columns from impurities.

Separation of the stabilized extract using a mobile phase comprising of water:acetonitrile:ammonium hydroxide solution mixed in a ratio of 70:30:0.01 (v:v:v).

Column—Hypersil® 150×4.6 mm i.d, C18 column (Phenomenex®, Torrence, Calif., USA).

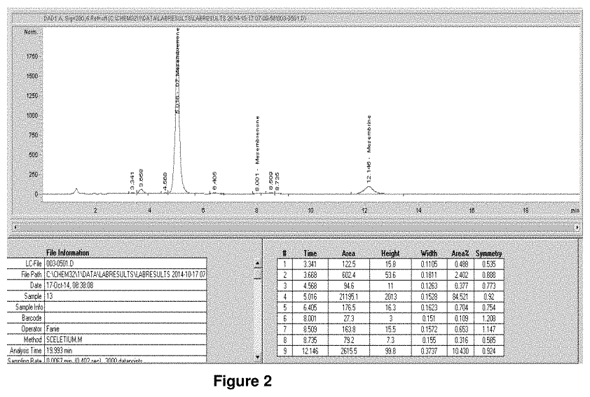

The results of the HPLC extraction are depicted in FIG. 2 of the accompanying figures.

Evaluation of the Effect of an Extract and Isolates from an Extract of Sceletium tortuosum on Normal and Cancerous Breast Epithelial Cells

In a preliminary test, the effect of an extract of Sceletium tortuosum (hereinafter PNP50) on the viability of normal and cancerous breast epithelial cells in culture was investigated.

Briefly, the procedure entailed 24 hour treatment of normal breast epithelial cells (MCF-12A) and breast epithelial carcinoma cells (MCF-7) with varying doses of the Sceletium extract named PNP50 (ranging from 0.25 to 1.5 mg/ml). At the end of this period, the mitochondrial viability of cells was assessed using the micro culture tetrazolium (MTT) assay method. Viability was measured and expressed relative to an appropriate control.

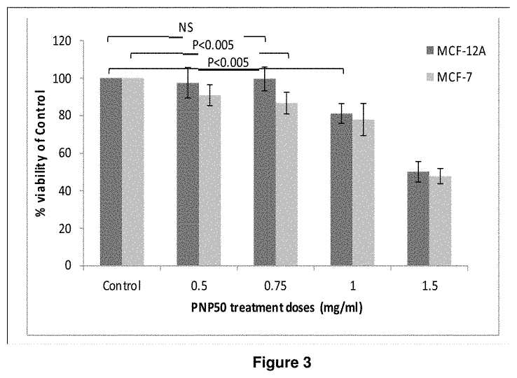

Results are illustrated in FIG. 3 of the accompanying figures and are expressed as the percentage of cells still viable after 24 hours. Bars are means of repeated experiments (a minimum of 5 repeats) and error bars indicate standard deviations. (For clarity, only statistical results relative to interpretation and conclusion are presented graphically.)

As is evident from the graph depicted in FIG. 3, extract PNP50 had no adverse effect on cell viability of normal breast epithelial cells at doses below 1 mg/ml, but became toxic to normal cells at a dose of 1 mg/ml, with increasing toxicity at higher doses. Carcinoma cells exhibited higher vulnerability to the extract when compared to normal cells, so that only 0.75 mg/ml was sufficient to decrease cell viability significantly.

As the results point to a positive effect of the tested extract as an anti-cancer modality, provided it is administered at the appropriate concentrations, further tests were conducted on specific candidates of the extracts on their effect on viability of normal and cancerous breast epithelial cells in culture.

Further investigations were conducted on isolates from the plant extracts, most notably Δ7 mesembrenone and mesembrenone, which were evaluated against an unknown non-alkaloid isolate from the extract. To calculate doses, the concentrations of the specific components for serving sizes of 15 mg, 30 mg and 60 mg per person per day for Δ7 mesembrenone, mesembrenone and non-alkaloids respectively were expressed as a fraction of total body fluid volume. In an attempt to account for digestive losses, these concentrations were halved, to reach cell-culture “equivalent” doses of 0.5 ug/ml, 1 ug/ml and 2 ug/ml respectively. Following standard laboratory practice, a range of doses around these was assessed. For clarity, only 3 concentrations per component are presented (doses of 10-fold smaller were also tested for all components, but were not different from lowest doses presented herein and thus omitted).

Briefly, the procedure itself entailed 24-hour treatment of confluent cell culture samples of normal breast epithelial cells (MCF-12A) and breast epithelial carcinoma cells (MCF-7) with varying doses of the test products. At the end of this period, the mitochondrial viability of cells was assessed using the XTT assay method. Viability was measured and expressed relative to an appropriate control.

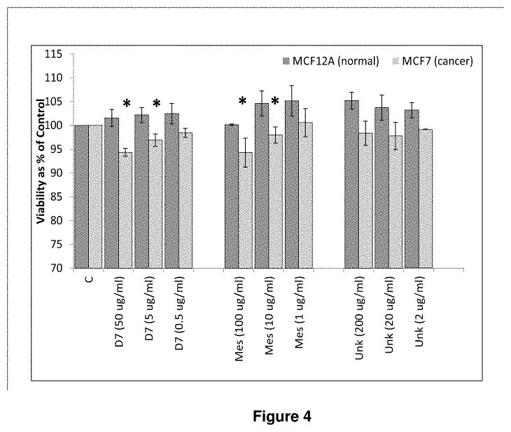

Results are illustrated in FIG. 4 of the accompanying drawings. Results are expressed as the percentage of viable cells after a 24 hour incubation with the test substances. Bars are means of repeated experiments (a minimum of 3 repeat experiments in triplicate) and error bars indicate standard deviations from the mean. Asterisk (*) indicate significant reductions in cancer cell viability.

Both Δ7 mesembrenone and mesembrenone showed clear dose-response effects on viability of cancer cells. Δ7 mesembrenone, which was administered at a dose 50% less than mesembrenone, appears to provide the most potent anti-cancer effect, while not affecting normal cell survival, even at the highest dose assessed. Mesembrenone also decreased survival of cancer cells, although the highest dose seemed to limit growth of normal cells to some extent as well. In terms of the unknown non-alkaloid fraction, none of the doses decreased cancer cell viability in comparison to control levels and certainly no dose-effect was evident.

Those skilled in the art will recognize that the strength of this anti-cancer effect is a rare occurrence—high effectivity with no adverse effects to normal cells. Given the promising results obtained for Δ7 mesembrenone and mesembrenone in this model, which is an estrogen-sensitive model, it is intended that the study will be followed up by an in vivo assessment using a rodent cancer model, as well as expansion of the investigation to a non-estrogen-sensitive model, such as colon cancer.

By way of non-limiting example only, pharmaceutical compositions of the invention are illustrated in the following examples.

### Example 1

A pharmaceutical strength oral capsule containing 102 mg of a composition of the invention—equal to 15 mg Δ7 mesembrenone, 4.29 mg mesembrine, 2.14 mg mesembrenone—and conventional pharmaceutical excipients

| | |
|---|---|
| Malto-dextrin (DE9) | 510 mg |
| Δ7 composition | 102 mg |
| Nu-FLOW ® (Rice concentrate) | 8.0 mg |
| Nu-Rice ® (Rice extract) | 0.5 mg |

A typical dose of the oral tablet composition is from 1 to 4 inclusive daily under the supervision of a medical practitioner.

### Example 2

A pharmaceutical strength oral capsule containing 15 mg Δ7 mesembrenone and conventional pharmaceutical excipients

| | |
|---|---|
| Malto-dextrin (DE9) | 597 mg |
| Δ7 mesembrenone (isolate) | 15.0 mg |
| Nu-FLOW ® (Rice concentrate) | 8.0 mg |
| Nu-Rice ® (Rice extract) | 0.5 mg |

A typical dose of the oral tablet composition is from 1 to 4 inclusive daily under the strict supervision of a medical practitioner.

### Example 3

Suppository formulation, formulated as disclosed in U.S. Pat. No. 2,538,127 A, containing 15 mg Δ7 mesembrenone, 4.29 mg mesembrine, 2.14 mg mesembrenone.

A typical dose of the composition is from 1 to 4 inclusive daily under the strict supervision of a medical practitioner.

### Example 4

A pharmaceutical strength viscous vape liquid containing 15 mg Δ7 mesembrenone.

| | |
|---|---|
| Δ7 mesembrenone (isolate) | 15 mg |
| USP Veg. Glycerine | 0.70 ml |
| USP Propelyne Glycol | 0.285 ml |

A 1 ml vape pod generally provides 122 vapes, the typical dose being from 1 to 20 vapes inclusive daily under the strict supervision of a medical practitioner.

### Example 5

A pharmaceutical strength oral capsule containing 20 mg Δ7 mesembrenone and conventional pharmaceutical excipients.

| | |
|---|---|
| Δ7 mesembrenone (isolate) | 20 mg |
| Olive Oil - [Dry Oil Powder] | 398.95 mg |
| (N-Zorbit 2144 DG) | |
| Tocopherol Acetate | 1 mg |
| Rosemary Leaf Extract | 0.05 mg |

A typical dose of the oral capsules is from 1 to 3 capsules inclusive daily under the strict supervision of a medical practitioner.

### Example 6

A pharmaceutical strength oral capsule containing 40 mg Δ7 mesembrenone and conventional pharmaceutical excipients.

| | |
|---|---|
| Δ7 mesembrenone (isolate) | 40 mg |
| Olive Oil - [Dry Oil Powder] | 378.95 mg |
| (N-Zorbit 2144 DG) | |
| Tocopherol Acetate | 1 mg |
| Rosemary Leaf Extract | 0.05 mg |

A typical dose of the oral capsules is from 1 to 2 capsules inclusive daily under the strict supervision of a medical practitioner.

### Example 7

A pharmaceutical strength topical balm containing 1625 mg Δ7 mesembrenone in a 150 ml jar with conventional topical non active ingredients.

| | |
|---|---|
| Δ7 mesembrenone (isolate) | 1625 mg |
| Shea Butter | 83.375 ml |
| Sesame | 30 ml |
| Bees Wax | 20 ml |
| Castor Oil | 15 ml |

A typical dose of the balm is from 1 to 8 1 ml applications inclusive daily under the strict supervision of a medical practitioner.

### Example 8

A pharmaceutical strength topical hydro-gel containing 600 mg Δ7 mesembrenone in a 30 ml bottle with conventional topical non active ingredients.

| | |
|---|---|
| Δ7 mesembrenone (isolate) | 600 mg |
| RO Water - Cold | 9.0 ml |
| RO Water - Hot | 5.00 ml |
| Glycerine | 8.50 ml |
| Guar Gum | 1.00 ml |
| Xanthan Gum | 1.00 ml |
| Sucragel | 4.90 ml |

A typical dose of the hydro-gel is from 1 to 4 1 ml applications inclusive daily under the strict supervision of a medical practitioner.

### Example 9

A pharmaceutical strength oral gummie containing 12.50 mg Δ7 mesembrenone in a 1.0 g piece of gum with suitable non active ingredients.

| | |
|---|---|
| Δ7 mesembrenone (isolate) | 12.50 mg |
| Gelatine | 96.00 mg |
| DI/RO Water | 195.50 mg |
| Sugar | 300.00 mg |
| Sorbitol Powder | 30.00 mg |
| Tartaric/Citric Acid | 20.00 mg |
| Essential Oil - Spearmint | 3.00 mg |
| Additional Flavour/Colour | 1.50 mg |
| Tocopherol Acetate | 1.00 mg |
| Rosemary Leaf Extract | 0.50 mg |
| Cornstarch - As Needed | |

A typical dose of the oral gum is from 1 to 4 gummies inclusive daily under the strict supervision of a medical practitioner.

1. A pharmaceutical composition comprising isolated Δ7 mesembrenone and a pharmaceutically acceptable carrier, together with other optional pharmaceutically acceptable excipients, wherein the pharmaceutical composition is in the form of a tablet, capsule, vape, topical balm or hydrogel, edible or chewable gum, or suppository.

2. The anti-cancer composition of claim 1, wherein the isolated Δ7 mesembrenone has a purity of at least 80%.

3. The anti-cancer composition claim 1, wherein the isolated Δ7 mesembrenone has a purity of at least 90%.

4. The anti-cancer composition of claim 1, having specific cytotoxicity on breast epithelial carcinoma cells (MCF-7).

5. A method of treating cancer comprising administering to a patient in need of such treatment a therapeutically effective amount of the pharmaceutical composition of claim 1.

6. The method of claim 5, wherein the cancer is breast cancer.

7. A unit dosage form comprising from about 1 mg to about 20 mg of Δ7 mesembrenone, wherein the unit dosage form is in the form of a tablet, capsule, vape, topical balm or hydrogel, edible or chewable gum, or suppository.

8. The unit dosage form of claim 7, comprising from about 5 mg to about 70 mg of Δ7 mesembrenone.

9. The unit dosage form of claim 7, comprising from about 10 mg to about 65 mg of Δ7 mesembrenone.

10. The unit dosage form of claim 7, comprising from about 15 mg to about 60 mg of Δ7 mesembrenone.
